(19) United States
(12) Patent Application Publication
(10) Pub. No.: US 2001/0053351 A1
(43) Pub. Date: Dec. 20, 2001

(54) Inhibition of GSK-3 beta

(76) Inventors: Klaus Hoeflich, Toronto (CA); James Woodgett, Toronto (CA); Juan Luo, Toronto (CA)

Correspondence Address: Pamela J. Sherwood, BOZICEVIC, FIELD & FRANCIS LLP, Suite 200, 200 Middlefield Road, Menlo Park, CA, 94025, US

(21) Appl. No.: 09/747,552

(22) Filed: Dec. 22, 2000

Related U.S. Application Data
(60) Provisional application No. 60/172,064, filed on Dec. 23, 1999.

(51) Int. Cl. A61K 38/19; A61K 31/00

(52) U.S. Cl. 424/85.1; 514/1

(57) ABSTRACT

The activity of NF-κB is modulated through the effects of GSK-3 on NF-κB activity. Inhibition or down-regulation of GSK-3 results in decreased NF-κB activity. Inappropriate activation of NF-κB has been linked to inflammation and hyperproliferative disorders. Development of modulatory strategies provide a novel therapeutic tool for the treatment or prevention of various diseases. Methods are also provided for enhanced killing of tumor cells through the sensitization action of GSK-3 inhibition, when administered in conjunction with apoptosis inducing ligands of TNFR1. Transgenic animals defective in GSK-3 function are also provided.

### BACKGROUND OF THE INVENTION

Perhaps the most difficult aspect of protein phosphorylation research is the linking of physiological substrates with particular protein kinases in order to reconstruct physiological pathways. For example, the enzyme glycogen synthase kinase-3 (GSK-3) was first studied for its role in intermediary metabolism. However, it has subsequently been found to have a role in numerous other processes, including transcriptional regulation.

Glycogen is the principal storage form of glucose in animal cells. It accumulates in electron-dense cytoplasmic granules and is synthesized by glycogen synthase (GS), the rate-limiting enzyme of glycogen deposition. Glycogen synthase kinase-3 (GSK-3) phosphorylates GS. Two nearly identical forms of GSK-3 exist: GSK-3 alpha and GSK-3 beta. Both are constitutively active in resting cells and their activity can be modulated by hormones and growth factors.

GSK-3 is also implicated in the regulation of many physiological responses in mammalian cells by phosphorylating substrates such as neuronal cell adhesion molecule, neurofilaments, synapsin I, and tau. GSK-3 phosphorylates several transcription factors including AP-1 and CREB, and the major nuclear pore protein p62. It also regulates PK1, a protein kinase required for maintaining the interphase state and for DNA replication in cycling Xenopus egg extracts. GSK-3 is inhibited by serine phosphorylation in response to insulin or growth factors, and is a substrate for protein kinase B (Cross et al. (1995) Nature 378(6559):785-9).

Nuclear Factor KB (“NF-κB”) is one of a family of transcription factors held in the cytoplasm of cells by inhibitors of the NF-κB protein (“IκB”). In response to extracellular stimuli, IκB is degraded, allowing NF-κB to migrate into the nucleus and activate select genes which elicit important immunological and proliferative responses. NF-κB plays an important role in autoimmune, inflammatory and cardiovascular disease through its regulation of cytokine genes such as IL-1, IL-2, IL-6, IL-8, TNF-α, along with genes that code for cell adhesion molecules and the COX-2 enzyme. Recent studies link NF-κB to increased cancer cell resistance to radiation and chemotherapy, and demonstrate the utility of NF-κB inhibitors to enhance the sensitivity of cancerous cells to these therapies.

IκB (inhibitor of NF-κB) kinase (IKK) phosphorylates IκB inhibitory proteins, causing their degradation, and subsequent activation of transcription factor NF-κB. IKK is composed of three subunits—IKKα and IKKβ, which are highly similar protein kinases, and IKKγ, a regulatory subunit. In mammalian cells, phosphorylation of two sites at the activation loop of IKKβ was essential for activation of IKK by tumor necrosis factor and interleukin-1, thus IKKβ is the target for proinflammatory stimuli. Once activated, IKKβ is autophosphorylated at a carboxyl-terminal serine cluster. Such phosphorylation decreases IKK activity and may prevent prolonged activation of the inflammatory response.

Tumor necrosis factor (TNF)-α has been shown to exert cytotoxic or cytostatic effects on tumor cells, but susceptibility to TNF-α varies among different types of cells. Activation of the type-1 TNF receptor (TNFR1) induces the formation of a signalling complex that contains TNF-receptor-associated-factor 2 (TRAF2), which binds NIK, a MAP kinase kinase kinase. Phosphorylation of the IKK component of the IκB kinase complex by NIK targets I-κB for degradation and induces NF-κB activation. An active NF-κB complex, such as the p50-p65 heterodimer, plays a crucial role in the progression of cell cycle in some malignancies. In some cases, refractoriness to TNF-α treatment can be prevented by inhibiting NF-κB activation (Otsuka et al. (1999) Cancer Research 59(7):4446-52).

The modulation of NF-kB activity is of great interest for the understanding of important physiological processes, and for the treatment of related disorders. For example, strategies to treat tumors with TNF-α and related proteins are of great interest, where it may be possible to “sensitize” resistant tumors to apoptosis. Methods of sensitization may permit the use of lower levels of the proteins, thereby reducing side effects. It may also permit the treatment of otherwise resistant tumors.

Relevant Literature

Barnes and Karin (1997) reviewed the role of NF-κB in chronic inflammatory diseases. They tabulate the stimuli that activate NF-κB and the proteins regulated by this transcription factor. They also discuss the effects of glucocorticoids on NF-κB and the therapeutic implications. The role of Akt serine threonine kinase in NF-κB activation by tumor necrosis factor is described by Ozes et al. (1999) Nature 401:82. A review of IκB-NF-κB structures is provided by Baeuerle (1998) Cell 95:729-731; and by Stancovski and Baltimore (1997) Cell 91:299-302.

TNF-α and IL-1 have been identified as important extracellular mediators that induce expression of a number of gene products involved in tissue inflammation (DiDonato et al. (1997) Nature 388:548-554). It is currently believed that their intracellular signals ultimately converge to induce a similar spectrum of gene products through activation of the nuclear transcription factor NF-κB.

A review of the TNF receptor superfamily may be found in Baker and Reddy (1998) Oncogene 17(25):3261-70. The tumor necrosis factor receptor (TNFR) superfamily represents a growing family, with over 20 members having been identified thus far in mammalian cells. These proteins share significant homologies in their extracellular ligand binding domains and intracellular effector (death) domains. Death signals seem to be associated with the activation of both the caspase and JUN kinase pathways. Gravestein and Borst (1998) Semin Immunol 10(6):423-34 also review this receptor superfamily.

### SUMMARY OF THE INVENTION

Compositions and methods are provided for modulating the activity of NF-κB through the effects of GSK-3 on NK-κB activity. Inhibition or down-regulation of GSK-3 results in decreased NF-κB activity. Inappropriate activation of NF-κB has been linked to inflammatory events associated with autoimmune arthritis, asthma, septic shock, lung fibrosis, glomerulonephritis, atherosclerosis, and AIDS. Development of modulatory strategies provide a novel therapeutic tool for the treatment or prevention of various diseases.

Methods are also provided for enhanced killing of tumor cells through the sensitization action of GSK-3 inhibition, when administered in conjunction with apoptosis inducing ligands of TNFR1, which include members of the tumor necrosis factor family, such as tumor necrosis factor-α. Interaction of these ligands with receptors causes the responding cell to undergo apoptosis. The sensitization by GSK-3 inhibitors allows increased killing at equivalent or lower doses of the TNFR1 ligands, and can sensitize otherwise resistant cells.

### BRIEF DESCRIPTION OF THE DRAWINGS

FIG. 1A shows the strategy for targeting the GSK-3β allele.

FIG. 1B shows a Southern blot analysis of mutant and wild-type embryos.

FIG. 1C shows a protein analysis from GSK-3β⁻/− cells using an antibody to total GSK-3.

FIG. 2A and FIG. 2B are examples of embryos that are heterozygous and homozygous for a targeted deletion on GSK-3β.

In FIG. 2C, pregnant females from GSK-3β⁺/−×GSK-3β⁺/− intercrosses were injected interperitoneally with 250 μg anti-murine TNF-α hamster monoclonal antibody or equal amounts of control. Liver section analysis was performed.

FIG. 3A demonstrates that GSK-3β-deficient cells have an increased sensitivity to TNF-induced apoptosis.

FIG. 3B shows the effect of administering the GSK-3β inhibitor, lithium, in wild-type cells.

FIG. 3C shows the sensitivity of GSK-3β−/− cells to various apoptotic stimuli.

FIG. 4A shows an electrophoretic mobility shift assay to determine the activation of NK-κB.

FIG. 4B shows a gel-shift analysis of wild-type or GSK-3β−/− cells treated with TNF-α. An immunoblot analysis of cytoplasmic IκB-α and nuclear 65 NF-κB is shown in FIG. 4C.

### DETAILED DESCRIPTION OF THE INVENTION

The inhibition of GSK-3 is found to affect the activity of the transcription factor NF-κB. NF-κB over-activity is associated with a number of pro-inflammatory conditions, which include autoimmune arthritis, asthma, septic shock, lung fibrosis, glomerulonephritis, atherosclerosis, and AIDS. By inhibiting GSK-3, the inflammatory immune responses mediated by NF-κB are ameliorated.

The inhibition of GSK-3 is also useful in sensitizing cells to apoptotic killing by ligands of TNFR1, e.g. TNF-α. The sensitization by GSK-3β inhibitors allows increased killing at equivalent or lower doses of the TNFR1 ligands, and can sensitize otherwise resistant cells.

In one aspect of the invention, transgenic cells and animals having a targeted disruption in the GSK-3β gene are provided, and may be used in diagnostic and screening assays. The homozygous mutation is lethal at the embryonic stage, and so this genotype is available in animal embryos, or as cell lines derived therefrom.

### Definitions

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which will be limited only by the appended claims.

GSK-3β: is a protein kinase found in a variety of organisms, including mammals. Two nearly identical forms of GSK-3 exist: GSK-3α and GSK-3β. Preferably, the GSK-3β is the target for inhibition with the subject methods. The human genetic sequences were characterised by Stambolic and Woodgett (1994) Biochem. J. 303:701-704. The amino acid sequence for GSK-3β can be accessed at Genbank, number P49841, and the corresponding nucleotide sequence at NM_{—}002093. For experimental and screening purposes, it may be desirable to use an animal model. For example, the rat GSK-3β sequence may be accessed at Genbank number P18266, and the mouse at Genbank accession number AAD39258.

GSK-3 inhibitors: as used herein, are compounds that directly or indirectly reduce the level of GSK-3 activity in a cell, by competitive or non-competitive enzyme inhibition; by decreasing protein levels, e.g. by a targeted genetic disruption, reducing transcription of the GSK-3 gene, increasing protein instability, etc. Inhibitors may be small organic or inorganic molecules, anti-sense nucleic acids, antibodies or fragments derived therefrom, etc.

Examples of direct inhibitors of GSK-3 protein include lithium (Klein et al. (1996) P.N.A.S. 93:8455-8459), which potently inhibits GSK-3 beta activity (K_{(I)}=2 mM), but is not a general inhibitor of other protein kinases. Valproic acid (VPA) is a potent broad-spectrum anti-epileptic with demonstrated efficacy in the treatment of bipolar affective disorder. VPA inhibits both GSK-3α and -3β, with significant effects observed at concentrations of VPA similar to those attained clinically (Chen et al.(1999) J Neurochem. 72:1327-30). Members of the class of compounds termed granulatimides, or didemnimides have been found to act as GSK-3 inhibitors (International patent application WO 99/47522). Other inhibitors may be found through screening combinatorial or other chemical libraries for the inhibition of GSK-3 activity.

Some indirect inhibitors of GSK-3 include wortmannin, which activates protein kinase B, resulting in the phosphorylation and inhibition of GSK-3. Isoproterenol, acting primarily through beta3-adrenoreceptors, decreases GSK-3 activity to a similar extent (approximately 50%) as insulin (Moule et al. (1997) J Biol Chem 272:7713-9). p70 S6 kinase and p90rsk-1 also phosphorylate GSK-3β, resulting in its inhibition.

Antisense nucleic acids or expression constructs may be used to inhibit the expression of GSK-3. Antisense molecules are used to down-regulate expression of TULP genes in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the GSK-3 gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise two or more different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996) Nature Biotechnology 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1996) supra. and Inouye et al. (1997) Ciba Found Symp. 209:224-33, and Pieken (1997) Ciba Found Symp. 209:218-22) Preferred oligonucleotides are chemically modified from the native phosphodiester structure, in order to increase their intracellular stability and binding affinity. A number of modifications have been described that alter the chemistry of the phosphodiester backbone, sugars or heterocyclic bases.

Antibodies may also be used to directly inhibit GSK-3 protein. Antibodies may be prepared in accordance with conventional ways, where the GSK-3 or a fragment thereof is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, New York, 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

NF-κB: Nuclear factor-κB (NF-κB) is a ubiquitous multicomponent protein complex that plays a major role in the regulation of many viral and cellular genes, and governs the expression of genes encoding cytokines, chemokines, growth factors, cell adhesion molecules, and some acute phase proteins in health and in various disease states. Inappropriate NFNF-κB activity is associated with pro-inflammatory conditions. NF-κB is activated by several agents, including cytokines, oxidant free radicals, inhaled particles, ultraviolet irradiation, and bacterial or viral products. Inhibition of NF-κB finds use in the treatment of autoimmune diseases characterized by the involvement of pro-inflammatory T cells, such as multiple sclerosis, experimental autoimmune encephalitis, rheumatoid arthritis and insulin dependent diabetes mellitus. Inhibition of NF-κB is also useful in sensitizing cells to TNFR1 mediated apoptosis, for the treatment of hyperproliferative conditions.

Transgenic Animals: For compound screening, determination of physiological pathways, etc., transgenic mice are provided. Such mice have a targeted disruption of the GSK-3β gene, such that there is substantially no active protein expressed from the targeted allele, hence a “knock-out”. Mice having a homozygous knock-out in this kinase die in late stage embryogenesis. The phenotype is similar to disruption of the RelA component of NF-κB and disruption of IκB kinase β. Like these models, the GSK-3 beta null embryos die of acute heptocyte apoptosis. The apoptotic effect of loss of GSK-3β is TNF-dependent since the death is inhibited by injection of anti-TNF antibodies, and mouse embryo fibroblasts mutant for both alleles of GSK-3β exhibit significantly higher sensitivity to TNF killing in culture.

The term “transgene” is used herein to describe genetic material that has been or is about to be artificially inserted into the genome of a mammalian cell, particularly a mammalian cell of a living animal. The transgene is used to transform a cell, meaning that a permanent or transient genetic change, preferably a permanent genetic change, is induced in a cell following incorporation of exogenous DNA. A permanent genetic change is generally achieved by introduction of the DNA into the genome of the cell. Vectors for stable integration include plasmids, retroviruses and other animal viruses, YACs, and the like. Of interest are transgenic mammals, e.g. cows, pigs, goats, horses, etc., and particularly rodents, e.g. rats, mice, etc.

Transgenic animals comprise an exogenous nucleic acid sequence present as an extrachromosomal element or stably integrated in all or a portion of its cells, especially in germ cells. Unless otherwise indicated, it will be assumed that a transgenic animal comprises stable changes to the germline sequence. During the initial construction of the animal, “chimeras” or “chimeric animals” are generated, in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

Transgenic animals fall into two groups, colloquially termed “knockouts” and “knockins”. In the present invention, knockouts have a partial or complete loss of function in one or both alleles of the endogenous GSK-3β gene. Knockins have an introduced transgene with altered genetic sequence and function from the endogenous gene. The two may be combined, such that the naturally occurring gene is disabled, and an altered form introduced.

In a knockout, preferably the target gene expression is undetectable or insignificant. A knock-out of a GSK-3β gene means that function of the GSK-3β enzyme has been substantially decreased so that expression is not detectable or only present at insignificant levels. This may be achieved by a variety of mechanisms, including introduction of a disruption of the coding sequence, e.g. insertion of one or more stop codons, insertion of a DNA fragment, etc., deletion of coding sequence, substitution of stop codons for coding sequence, etc. In some cases the exogenous transgene sequences are ultimately deleted from the genome, leaving a net change to the native sequence. Different approaches may be used to achieve the “knock-out”. A chromosomal deletion of all or part of the native gene may be induced, including deletions of the non-coding regions, particularly the promoter region, 3′ regulatory sequences, enhancers, or deletions of gene that activate expression of GSK-3β genes. A functional knock-out may also be achieved by the introduction of an antisense construct that blocks expression of the native genes (for example, see Li and Cohen (1996) Cell 85:319-329). “Knock-outs” also include conditional knock-outs, for example where alteration of the target gene occurs upon exposure of the animal to a substance that promotes target gene alteration, introduction of an enzyme that promotes recombination at the target gene site (e.g. Cre in the Cre-lox system), or other method for directing the target gene alteration postnatally.

A “knock-in” of a target gene means an alteration in a host cell genome that results in altered expression or function of the native GSK-3β gene. Increased (including ectopic) or decreased expression may be achieved by introduction of an additional copy of the target gene, or by operatively inserting a regulatory sequence that provides for enhanced expression of an endogenous copy of the target gene. These changes may be constitutive or conditional, i.e. dependent on the presence of an activator or represser.

The exogenous gene is usually either from a different species than the animal host, or is otherwise altered in its coding or non-coding sequence. The introduced gene may be a wild-type gene, naturally occurring polymorphism, or a genetically manipulated sequence, for example having deletions, substitutions or insertions in the coding or non-coding regions. The introduced sequence may encode a GSK-3β polypeptide, or may utilize the GSK-3β promoter operably linked to a reporter gene. Where the introduced gene is a coding sequence, it is usually operably linked to a promoter, which may be constitutive or inducible, and other regulatory sequences required for expression in the host animal. By “operably linked” is meant that a DNA sequence and a regulatory sequence(s) are connected in such a way as to permit gene expression when the appropriate molecules, e.g. transcriptional activator proteins, are bound to the regulatory sequence(s).

Specific constructs of interest, but are not limited to, include anti-sense GSK-3β, which will block native GSK-3β expression, expression of dominant negative GSK-3β mutations, and over-expression of a GSK-3β gene. A detectable marker, such as lac Z may be introduced into the locus, where upregulation of expression will result in an easily detected change in phenotype. Constructs utilizing the GSK-3β promoter region, in combination with a reporter gene or with the coding region are also of interest.

A series of small deletions and/or substitutions may be made in the GSK-3β gene to determine the role of different exons in kinase activity, transcriptional regulation, etc. By providing expression of GSK-3β protein in cells in which it is otherwise not normally produced, one can induce changes in cell behavior.

DNA constructs for homologous recombination will comprise at least a portion of the GSK-3β gene with the desired genetic modification, and will include regions of homology to the target locus. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al. (1990) Methods in Enzymology 185:527-537.

For embryonic stem (ES) cells, an ES cell line may be employed, or embryonic cells may be obtained freshly from a host, e.g. mouse, rat, guinea pig, etc. Such cells are grown on an appropriate fibroblast-feeder layer or grown in the presence of appropriate growth factors, such as leukemia inhibiting factor (LIF). When ES cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting litters screened for mutant cells having the construct. By providing for a different phenotype of the blastocyst and the ES cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture.

TNFR1 Ligand: TNFR1 ligands, as defined herein, refer to compounds, usually polypeptide compounds, that bind to the mammalian cell surface receptor TNFR1, which comprises a death domain, and on binding so deliver a signal for apoptosis to the cell. The intracellular protein interactions triggered by these receptors can be attributed to binding interactions of the death domain, which is homologous to an approximately 80 amino acid domain near the C-terminus of TNF-R1, and is responsible for signalling cytotoxicity (Kitson et al. (1996) Nature 384:372-5). Exemplary of a TNFR1 ligand is TNF itself, but other compounds that bind to the receptor and activate are also suitable.

Other molecules that interact with TNFR1 may be used in the subject methods. Such ligands will specifically bind to the extracellular domain of the receptor, and compete with the cognate ligand for binding. Ligands will also activate signalling through the death domain to activate apoptosis. Candidate ligands are screened for their ability to meet this criteria. Assays to determine affinity and specificity of binding are known in the art, including competitive and non-competitive assays. Assays of interest include ELISA, RIA, flow cytometry, etc. Binding assays may use purified or semi-purified protein, or alternatively may use cells that express TNFR1, e.g. cells transfected with an expression construct for TNFR1, etc. As an example of a binding assay, purified receptor protein is bound to an insoluble support, e.g. microtiter plate, magnetic beads, etc. The candidate ligand and soluble, labelled TNFR1 are added to the cells, and the unbound components are then washed off. The ability of the ligand to compete for receptor binding is determined by quantitation of bound, labelled ligand. A functional assay that detects apoptosis may be used for confirmation.

Suitable ligands in addition to TNF-α and variants thereof, include peptides, small organic molecules, peptidomimetics, antibodies, or the like. Antibodies may be polyclonal or monoclonal; intact or truncated, e.g. F(ab′)₂, Fab, Fv; xenogeneic, allogeneic, syngeneic, or modified forms thereof, e.g. humanized, chimeric, etc.

Pharmaceutical Formulation: The GSK-3 inhibitor may be combined with a pharmaceutically acceptable carrier, which term includes any and all solvents, dispersion media, coatings, anti-oxidant, isotonic and absorption delaying agents and the like. The use of such media and agents for pharmaceutically active substances is well known in the art. Except insofar as any conventional media or agent is incompatible with the active ingredient, its use in the therapeutic compositions and methods described herein is contemplated. Supplementary active ingredients can also be incorporated into the compositions.

The formulation may be prepared for use in various methods for administration. The formulation may be given orally, by inhalation, or may be injected, e.g. intravascular, intratumor, subcutaneous, intraperitoneal, intramuscular, etc.

The dosage of the therapeutic formulation will vary widely, depending upon the nature of the disease, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc. to maintain an effective dosage level. In some cases, oral administration will require a higher dose than if administered intravenously.

The GSK-3 inhibitor can be incorporated into a variety of formulations for therapeutic administration. More particularly, the complexes can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the GSK-3 inhibitor can be achieved in various ways. The GSK-3 inhibitor may be systemic after administration or may be localized by the use of an implant that acts to retain the active dose at the site of implantation.

For use in the subject methods, the GSK-3 inhibitor may be formulated with other pharmaceutically active agents, particularly other anti-metastatic, anti-tumor or anti-angiogenic agents. Angiostatic compounds of interest include angiostatin, endostatin, carboxy terminal peptides of collagen alpha (XV), etc. Cytotoxic and cytostatic agents of interest include adriamycin, alkeran, Ara-C, BICNU, busulfan, CNNU, cisplatinum, cytoxan, daunorubicin, DTIC, 5-FU, hydrea, ifosfamide, methotrexate, mithramycin, mitomycin, mitoxantrone, nitrogen mustard, velban, vincristine, vinblastine, VP-16, carboplatinum, fludarabine, gemcitabine, idarubicin, irinotecan, leustatin, navelbine, taxol, taxotere, topotecan, etc.

Methods of Use

Inhibition of NF-κB

The activity of NF-κB in inhibited through a decrease in GSK-3 activity, particularly GSK-3β activity. The cellular level of GSK-3 may be reduced by competitive or noncompetitive direct inhibition of the enzymatic activity, indirect inhibition of the enzymatic activity, or reductions in protein expression, stability, etc.

Increased activity of NF-κB is associated with inflammation. Inflammation is a localized protective response mounted by tissues in response to injury, infection, or tissue destruction resulting in the destruction of the infectious or injurious agent and isolation of the injured tissue. In many human diseases with an inflammatory component, the normal, homeostatic mechanisms which attenuate the inflammatory responses are defective, resulting in damage and destruction of normal tissue.

The GSK-3 activity in a targeted cell is reduced through administration of an inhibitor at a dose that reduces inflammation in inflammatory disorders or diseases, including asthma, juvenile diabetes mellitus, myasthenia gravis, Graves' disease, rheumatoid arthritis, allograft rejection, inflammatory bowel disease, multiple sclerosis, psoriasis, lupus erythematosus, systemic lupus erythematosus, diabetes, multiple sclerosis, contact dermatitis, rhinitis and various allergies.

For example, GSK-3 inhibitors may be administered in the treatment of asthma. Asthma is characterised by the hyper-responsiveness of the tracheobronchial tree to various stimuli such as allergens, exercise, temperature, chemicals and spores. The most common asthma is atopic or allergic asthma and involves an immediate response due to mast cell histamine release and release of inflammatory modulators which recruit eosinophils, neutrophils and lymphocytes. The acute reaction results in bronchoconstriction, edema, increased mucus secretion, flushing, and in some cases hypotension. A late phase reaction four to eight hours later, lasting up to 24 hours, occurs due to the presence of the large population of recruited inflammatory cells which release further mediators of bronchoconstriction leading to edema and epithelial damage.

The GSK-3 inhibitors are also useful for preventing, inhibiting and/or ameliorating inflammatory and immune reactions associated with systemic lupus erythematosus (SLE). SLE is a classical multisystem autoimmune disease characterized by the presence of tissue damage due to self antigen directed antibodies. Autoantibodies bound to antigens in various organs lead to complement-mediated and inflammatory cell mediated tissue damage. Skin, connective tissue, blood vessels, and joints are all effected in this chronic, remitting and relapsing disease, but kidney failure due to antibody mediated glomerulonephritis is the main life-threatening complication. The present invention is useful in treating other autoimmune disorders such as scleroderma, various forms of vasculitis, inflammatory autoimmune myositis, and autoimmune thyroiditis.

The compositions and methodologies of the present invention are also efficacious in the treatment of multiple sclerosis (MS). M.S. is characterized by the penetration of the blood-brain barrier by circulating leukocytes, leading to demyelination in various parts of the brain, impaired nerve conduction and, ultimately, paralysis. Certain T cell clones reactive to myelin basic protein localize in the central nervous system and initiate inflammation.

The present invention is also efficacious for treatment of different forms of inflammatory arthritis. There are many different types of arthritis clinically recognized, the most common being rheumatoid arthritis. However, the inflammatory pathway relevant to the pathogenesis of rheumatoid arthritis is also likely relevant to the pathogenesis of other types of arthritis e.g. osteo, psoriatic and spondyloarthropathies since the synovial pathologies in all these forms of arthritis is in many cases, the same. The present invention is useful for treating many other clinical conditions involving inflammatory processes. For example, inflammatory bowel diseases including Crohn's disease and ulcerative colitis are spontaneous chronic inflammations of the gastrointestinal tract which involve activation of inflammatory cells whose products cause tissue injury. Neutrophils, eosinophils, mast cells, lymphocytes and macrophages contribute to the inflammatory response.

Psoriasis, which is characterized by, among other symptoms, epidermal hyperplasia/thickening and minute microabcesses of neutrophils in the upper epithelial layers of the dermis, is also treatable by the compositions and methodologies of the present invention. Psoriasis is believed to be caused by an autoimmune inflammatory response to a set of antigens in the skin. An increased autologous T cell response is seen in cells derived from a psoriatic lesion.

The present invention is also directed to treatment of systemic shock and many resultant clinical conditions associated therewith. Systemic shock often occurs as a complication of severe blood loss, severe localized bacterial infection, ischemia/reperfusion trauma and is a major cause of death in intensive care units. Most cases of septic shock are induced by endotoxins from gram negative bacilli or toxins from gram positive cocci bacteria. The release of LPS in the bloodstream causes release of inflammatory mediators (inflammatory cytokines, platelet activating factor, complement, leukotrienes, oxygen metabolites, and the like) which cause myocardial dysfunction, vasodilation, hypotension, endothelial injury, leukocyte adhesion and aggregation, disseminated intravascular coagulation, adult respiratory distress syndrome (ARDS), liver, kidney and central nervous system (CNS) failure. Shock due to blood loss also involves inflammatory mediator release. In each case, inflammatory responses are induced at the original site of trauma, and also in the vasculature and remote vascularized sites.

Inflammatory response damage also occurs in glomerulonephritis as well as tubule disease. Infiltration of inflammatory cells (especially macrophages) is linked to proteinuria accompanied histologically by hypercellularity and crescent formation in glomeruli. Over a longer term, the infiltration of inflammatory cells is associated with accumulation of extracellular matrix and sclerosis and chronic compromise of renal function. The present invention is also efficacious in treating glomerulonephritis and tubule disease.

The subject therapy will desirably be administered during the presymptomatic or preclinical stage of the disease, and in some cases during the symptomatic stage of the disease. Early treatment is preferable, in order to prevent the loss of function associated with inflammatory tissue damage. The presymptomatic, or preclinical stage will be defined as that period not later than when there is inflammatory cell involvement at the site of disease, e.g. islets of Langerhans, synovial tissue, thyroid gland, etc., but the loss of function is not yet severe enough to produce the clinical symptoms indicative of overt disease. Inflammatory cell involvement may be evidenced by the presence of elevated numbers of cells at the site of disease, the presence of autoantigen specific cells, the release of performs and granzymes at the site of disease, response to immunosuppressive therapy, etc.

Mammalian species susceptible to inflammatory conditions include canines and felines; equines; bovines; ovines; etc. and primates, particularly humans. Animal models, particularly small mammals, e.g. murine, lagomorpha, etc. may be used for experimental investigations. Animal models of interest include those involved with cytokine production and response e.g. TNF-α, IL-1, NF-κB, GSK-3, etc. Other uses include investigations where it is desirable to investigate a specific effect in the absence of NF-κB mediated inflammation.

Treatment of Hyperproliferative Conditions and Sensitization to TNFR1 Mediated Apoptosis

A therapy of GSK-3 inhibitor, or combined therapy of GSK-3 inhibitor and TNFR1 ligand is administered to a host suffering from a susceptible hyperproliferative disorders, which may include psoriasis, arthritis, inflammation, cancer, etc. Administration may be topical, localized or systemic, depending on the specific disease. The compounds are administered at an effective dosage that over a suitable period of time substantially reduces the tumor cell burden, while minimizing any side-effects, usually killing at least about 25% of the tumor cells present, more usually at least about 50% killing, and may be about 90% or greater of the tumor cells present. It is contemplated that the composition will be obtained and used under the guidance of a physician for in vivo use. To provide the synergistic effect of a combined therapy, the active agents can be delivered together or separately, and simultaneously or at different times within the day.

Tumors known susceptible to induction of apoptosis include carcinomas, e.g. colon, prostate, breast, melanoma, ductal, endometrial, stomach, dysplastic oral mucosa, invasive oral cancer, non-small cell lung carcinoma, transitional and squamous cell urinary carcinoma, etc.; neurological malignancies, e.g. neuroblastoma, gliomas, etc.; hematological malignancies, e.g. childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

The susceptibility of a particular tumor cell to killing with the combined therapy may be determined by in vitro testing, as detailed in the experimental section. Typically a culture of the tumor cell is combined with a combination of a DDL and a diterpenoid triepoxide at varying concentrations for a period of time sufficient to allow the active agents to induce apoptosis, usually between about one hour and one week. For in vitro testing, cultured cells from a biopsy sample of the tumor may be used. The viable cells left after treatment are then counted.

The dose will vary depending on the specific agents utilized, type of tumor, patient status, etc., at a dose sufficient to substantially ablate the tumor cell population, while maintaining patient viability. In some cases therapy may be combined with stem cell replacement therapy to reconstitute the patient hematopoietic function. Treatment will generally be continued until there is a substantial reduction, e.g. at least about 50%, decrease in the tumor burden, and may be continued until there are essentially no tumor cells detected in the body.

Drug Screening

The transgenic mice of the invention may be used to assess the effect of a compound on GSK-3 inhibited cells, and for the determination of pathways relating to GSK-3. Through use of the subject transgenic animals or cells derived therefrom, one can identify ligands or substrates that replace, bind to, modulate, antagonize or agonize GSK-3. Screening to determine drugs that lack effect on this kinase is also of interest. Since the catalytic domains of GSK-3alpha and GSK-3beta have substantial identity, either protein may be used for in vitro screening assays.

A wide variety of assays may be used for this purpose, including determination of the localization of drugs after administration, labeled in vitro protein-protein binding assays, protein-DNA binding assays, electrophoretic mobility shift assays, immunoassays for protein binding, and the like. Depending on the particular assay, whole animals may be used, or cell derived therefrom. Cells may be freshly isolated from an animal, or may be immortalized in culture.

The term “agent” as used herein describes any molecule, e.g. protein or pharmaceutical, with the capability of affecting the biological action of GSK-3 and its signaling pathway. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the array” includes reference to one or more arrays and equivalents thereof known to those skilled in the art, and so forth. All technical and scientific terms used herein have the same meaning as commonly understood to one of ordinary skill in the art to which this invention belongs unless clearly indicated otherwise.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### EXAMPLE 1

Effect of NF-κB Activation in the Knockout Mouse Embryos by Gel Shift Assay and Reporter Assay

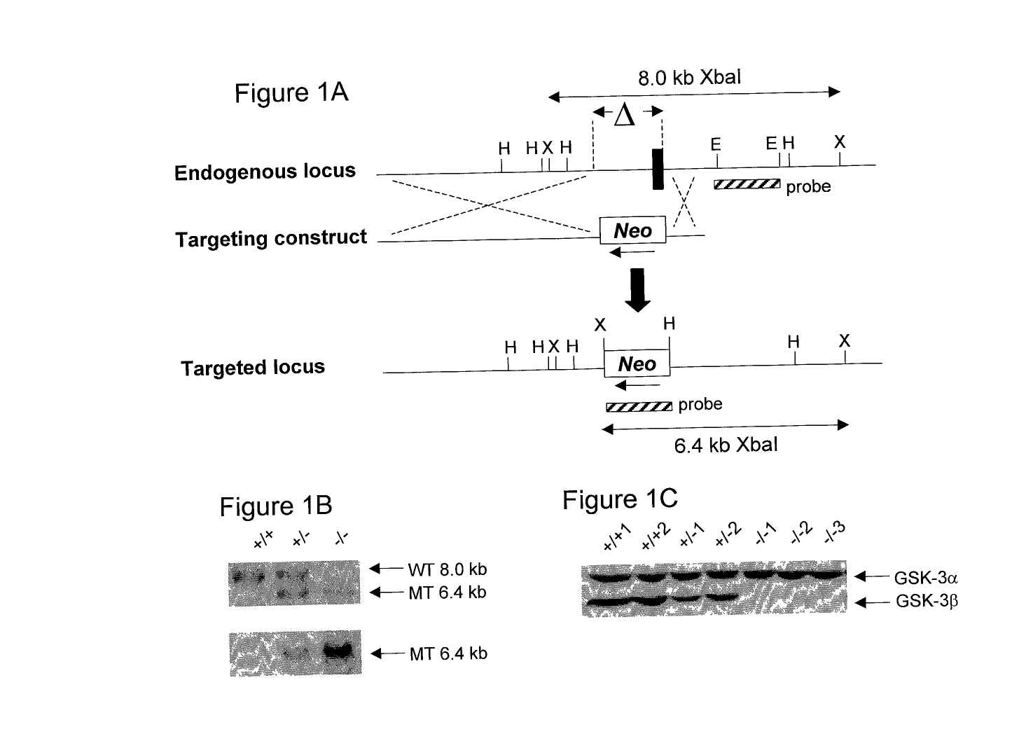

To investigate the role of GSK-3 in mammalian development, we disrupted the GSK-3β gene in murine 129J embryonic stem (ES) cells using a targeting vector in which the exon encoding the ATP-binding loop was deleted (FIG. 1A). Chimeric mice derived from two independent heterozygous ES clones were back-crossed to C57BL/6J mice, and heterozygous mice were crossed to generate homozygous mutant offspring. In addition to tail DNA genotyping, the null mutation of GSK-3β was confirmed by Southern blot (FIG. 1B) and Western blot (FIG. 1C) analyses of embryonic fibroblasts (EFs) derived from mice on day 12.5 of gestation (E12.5). Although GSK-3β^{+/-} male and female mice were healthy and fertile, upon intercrossing they did not give rise to live GSK-3β^{-/-} progeny.

FIG. 1A shows a portion of the mouse GSK-3β wild-type locus (top) showing exons (solid box) and a 8.0 kb Xbal fragment in the wild-type allele. The targeting vector (middle) was designed to replace an exon encoding a portion of the GSK-3β kinase domain, including the ATP interacting lysine residue (Lys85). The mutated GSK-3β locus (bottom) contains a 6.4 kb Xbal fragment. The positions of the probes used for Southern blot analysis are shown (hatched boxes). X, E and H represent Xbal, EcoRI and HindIII, respectively. (B) Southern blot analysis of wild-type and mutant embryonic fibroblast cell DNA. Genomic DNA from wild-type (lane 1), GSK-3β^{+/-} (lane 2), and GSK-3β^{-/-} EF cell clones (lane 3) were digested with Xbal and hybridized to the flanking probe. The 8.0 kb wild-type fragment and the 6.4 kb mutant fragment are indicated. (C) GSK-3β protein is undetectable in GSK-3β^{-/-} EF cells. Total protein (40 μg) extracted from independent EF clones was analyzed for the expression of the GSK-3β gene using an antibody to total GSK-3 (mouse monoclonal, Upstate Biotechnology). The upper band is GSK-3 α. The genotypes are as indicated.

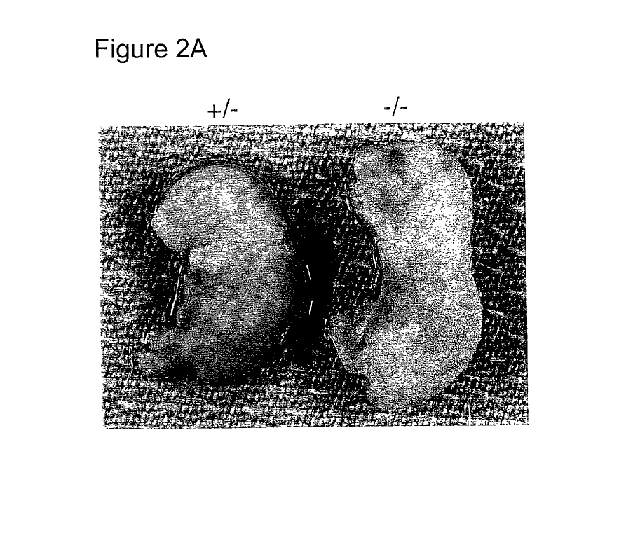

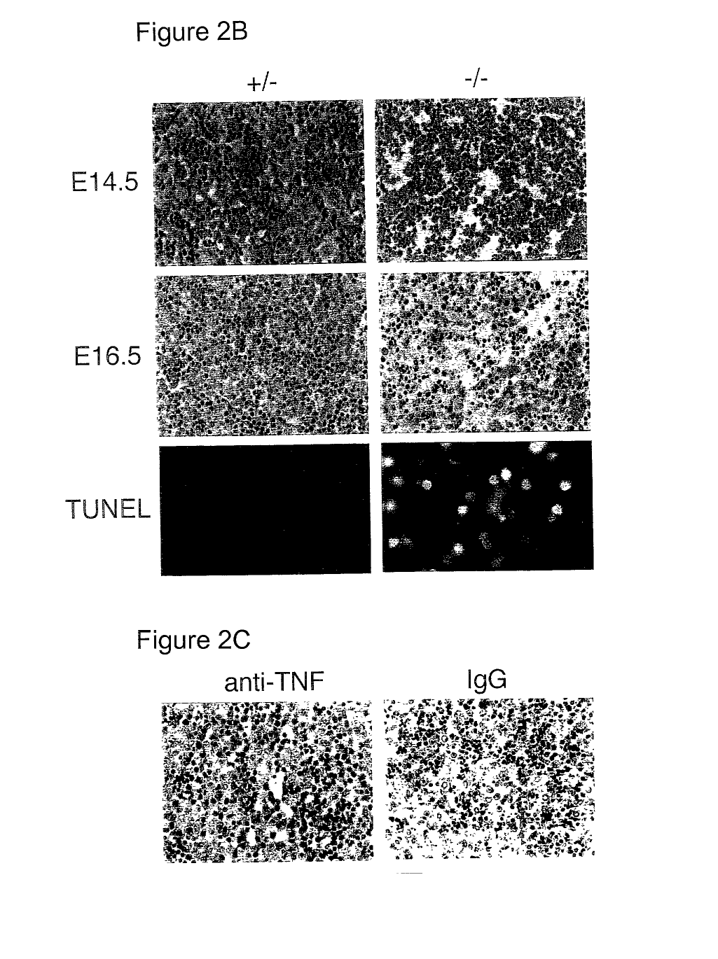

We thus analyzed embryos from timed pregnancies of GSK-3β^{+/-} intercrosses. Before E12.5, the Mendelian ratio of null embryos was normal and most GSK-3β^{-/-} embryos showed no morphological abnormality. However, between E13.5-14.5, GSK-3β^{-/-} embryos appeared pale and non-viable (FIG. 2A). Histological examination of E13.5 GSK-3β^{+/-} and GSK-3β^{-/-} embryos demonstrated multifocal hemorrhagic degeneration in the livers of homozygous embryos (FIG. 2B). Examples of E14.5 wild-type and GSK-3β^{-/-} embryos are shown in (A) and (B), respectively. GSK-3β^{-/-} embryos at this stage showed haemorrhaging of the liver. No differences were detected between GSK-3β wild-type and heterozygous embryos. The genotypes of the embryos were later determined by Southern and Western blot analyses. For histology, embryos were fixed with 10% neutral buffered formalin, embedded in paraffin and four micron transverse sections were stained with hematoxylin and eosin. GSK-3β^{-/-} embryos exhibit significantly more apoptotic nuclei (arrowheads) than their GSK-3β^{+/-} littermates. The scale bar indicates 100 μm in (C) and (D). At this age, increased TUNEL-positive cells are observed in GSK-3β^{-/-} mice (F) compared to controls (E). Terminal deoxynucleotidyl transferase-mediated dUTP nick end labelling (TUNEL) assays were performed according to the manufacturer's instructions (Boehringer Mannheim) and sections counterstained with hematoxylin. In (G), (H) and (I), pregnant females from GSK-3β^{+/-}×GSK-3β^{+/-} intercrosses were injected interperitoneally with 250 μg anti-murine TNF-α hamster monoclonal antibody or equal amount of control hamster IgG (Genzyme). Liver section analysis was performed on GSK-3β null genotyped embryos as described above.

In these areas, the hepatocytes showed pyknosis and karyorrhexis consistent with cells that were undergoing apoptosis. This was confirmed by terminal deoxytransferase-mediated deoxyuridine triphosphate nick-end labelling (TUNEL) assay which revealed numerous positively-stained nuclei (FIG. 2B). GSK-3β^{-/-} embryos derived from the two original GSK-3β heterozygous ES cell clones were indistinguishable in phenotype. Fetal liver transfer experiments demonstrated that hematopoietic cells from GSK-3β^{-/-} embryos were capable of reconstituting hematopoiesis in lethally irradiated mice. These results suggested that hepatocyte apoptosis is likely the major cause of lethality in the GSK-3β^{-/-} embryos.

This phenotype is remarkably similar to that of IKKβ and RelA-deficient mice which has been demonstrated to result from increased sensitivity to TNF-α.

### EXAMPLE 2

Inhibition of Endogenous TNF-α

To inhibit endogenous TNF-α, we injected anti-murine TNF-α monoclonal hamster antibody (or hamster immunoglobins as a control) into the peritoneum of pregnant heterozygous females (sired by heterozygous males) on E10.5 or E11.5. The females were sacrificed and embryos collected on E14.5. From the 3 females treated with anti-TNF-αantibody injection, 11 embryos (42% of total) were identified as GSK-3β^{-/-}. As determined by gross morphology and histological analysis of the liver, substantial protection and hepatocyte rescue of the anti-TNF-α-injected embryos was observed, as compared to control embryos (FIG. 2C). These results point to an unexpected role for GSK-3β in suppressing TNF-mediated apoptosis.

### EXAMPLE 3

Gene Expression Profiles of Mouse Embryo Fibroblasts Treated with or without TNF-α, with and without Expression of GSK3-beta

To probe the mechanism by which GSK-3β impacted TNF cytotoxicity, immortalized mouse embryonic fibroblast (MEF) cell lines from two wild-type (+/−1 and +/−2), two GSK-3β heterozygous (+/−1 and +/−2) and two GSK-3β homozygous mutant (−/−1 and −/−2) embryos were derived from embryos. Immunoblotting of cell lysates from these cell lines confirmed the loss of GSK-3β expression in the two GSK-3β^{-/-} cell lines (FIG. 1C), whereas expression of proteins important for TNF signaling, such as TNF-R1 and IKKβ were equivalent.

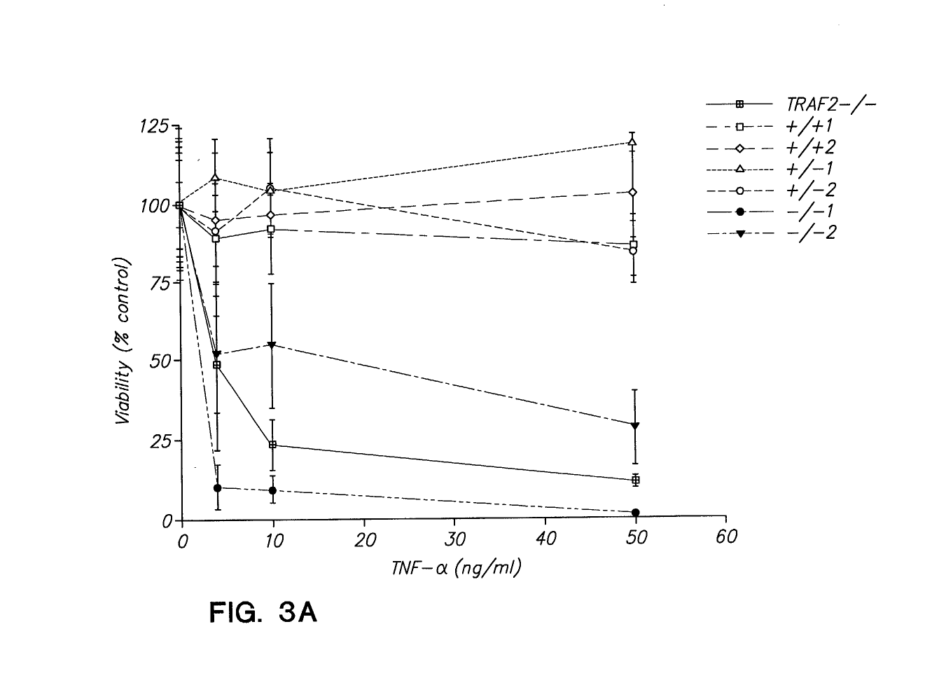

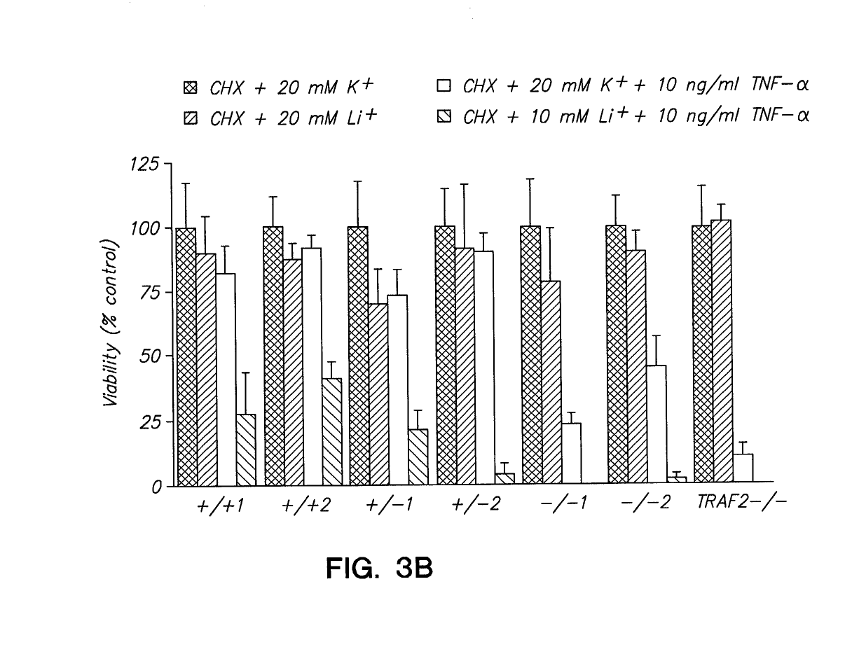

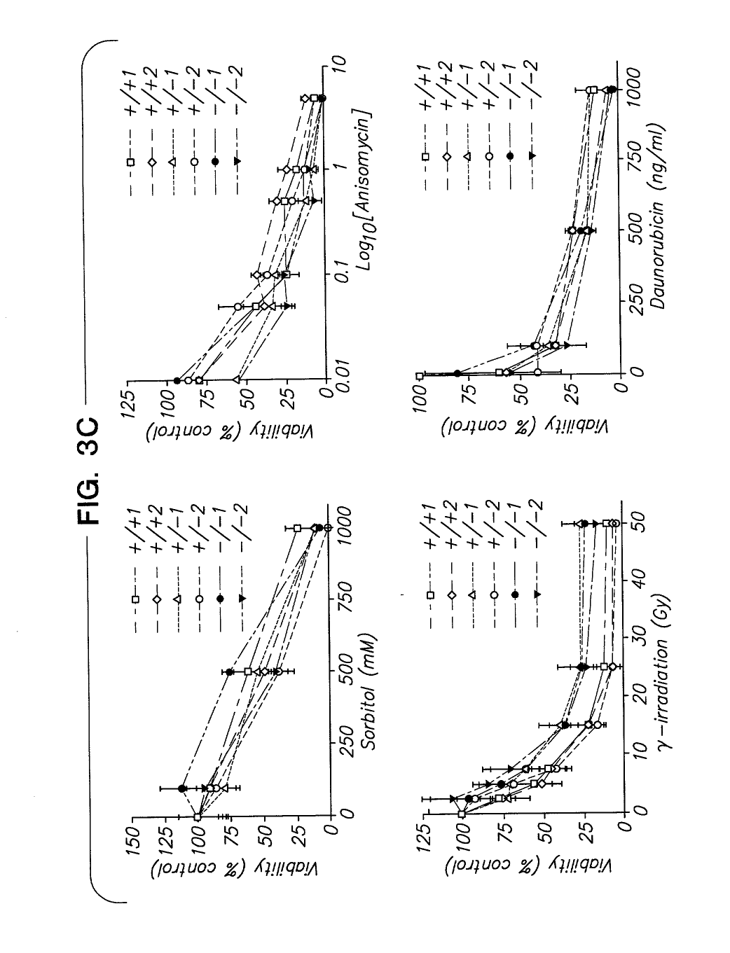

We next examined the sensitivity of the lines to TNF-induced cell death. As measured by viability dye staining, wild-type and GSK-3β-heterozygous cells were relatively unaffected by exposure to TNF-α (4, 10 or 50 ng/ml) and cycloheximide (0.25 μg/ml; FIG. 3A). However, significantly fewer GSK-3β^{-/-} cells remained viable 24 hr following maximum TNF-α treatment (2% and 28% for the −/−1 and −/−2 cell lines, respectively). As a reference, TNF receptor-associated factor 2 (TRAF2) homozygous mutant MEFs were used in the same assay. TRAF2^{-/-} cell survival in our hands was consistent with previous reports (˜20% at 10 ng/ml TNF-α and 0.25 μg/ml cycloheximide)(Yeh et al. (1997) Immunity 7:715-725)

### EXAMPLE 4

Effect of Li and other GSK-3beta Inhibitors upon Gene Expression.

Lithium ions are effective inhibitors of GSK-3α and β both in vitro and in intact cells and have been reported to exacerbate TNF cytotoxicity (Stambolic et al., supra.) Cells were incubated with 20 mM lithium (or 20 mM potassium as a control) during treatment with cycloheximide and an intermediate (10 ng/ml) TNF-α concentration. With the exception of the combination of lithium/TNF-α/cycloheximide, wild-type and heterozygous cell lines were unaffected by all other combinations of treatments (FIG. 3B). Figure (A) Twenty-four hours after the indicated TNF-α (4, 10 or 50 ng/ml) plus cycloheximide (0.25 μg/ml) treatment, viable cells were determined by negative staining with trypan blue. Viability is expressed normalized to untreated controls. Independently derived GSK-3β^{+/+} EF cell clones are indicated by +/+1 and +/+2; GSK-3β^{+/-} by +/−1 and +/−2; and GSK-3β^{-/-} by −/−1, and −/−2. (B) Lithium potentiates the TNF killing in wild-type cells. Cells were uniformly treated with cycloheximide (CHX, 0.25 μg/ml). In addition, 20 mM potassium (K⁺, solid bars), 20 mM lithium (Li⁺, dotted bars), 20 mM K⁺/10 ng/ml TNF-α (open bars) or 20 mM Li⁺/10 ng/ml TNF-α (gray bars) was added. (C) Sensitivity of GSK-3β^{-/-} EF cells to various apoptotic stimuli. EF cells were stimulated with anisomycin (0-5 μg/ml), sorbitol (0-1000 mM), daunorubicin (0-1000 ng/ml), and γ-irradiation (0-50 Gy). All experiments were repeated at least three times, and the data (as a percentage) is shown as the mean ± standard error.

Consistent with the inhibitory effect of lithium on GSK-3, exposure to lithium, TNF-α and cycloheximide together yielded statistically significant decreases in cell viability both the wild type and GSK-3β heterozygous lines. To assess the specificity of the apoptotic sensitivity to TNF upon disruption of GSK-3β function, GSK-3β^{-/-} cells were treated with a range of apoptotic stimuli (anisomycin, sorbitol, daunorubicin or γ-irradiation). The GSK-3β-deficient cells exhibited normal susceptibility to these stimuli (FIG. 3C). Taken together, the aforementioned results indicate that the presence of GSK-3β is required for selective protection of cells from TNF-α.

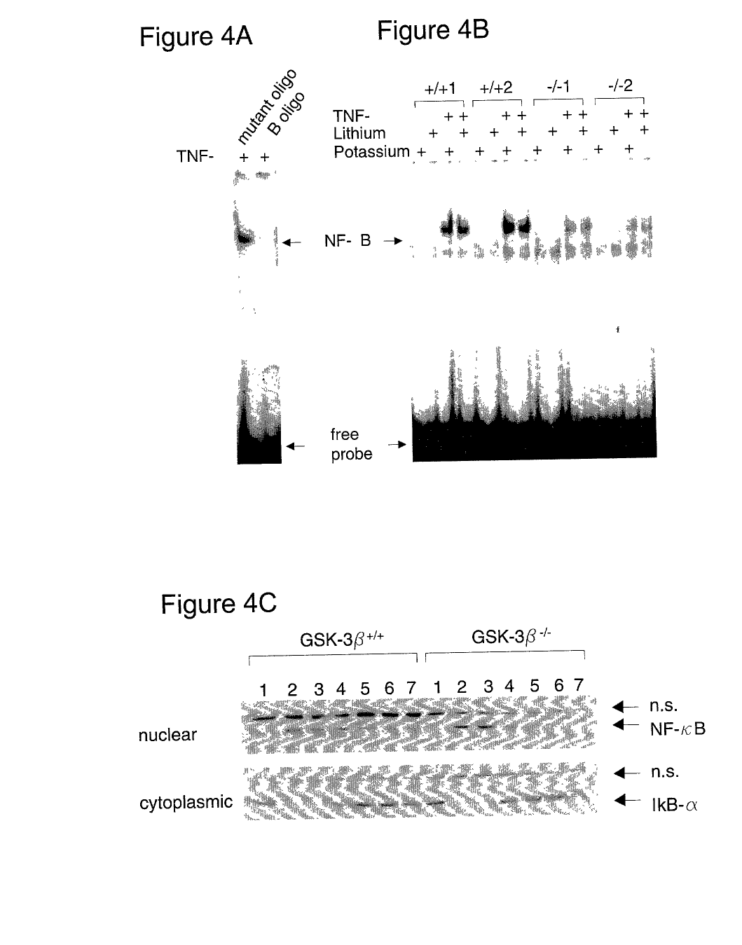

Several studies have demonstrated that activation of the transcription factor NF-κB is essential for suppression of TNF-induced apoptosis (Beg et al. (1996) Science 274:782-784). We reasoned that the TNF sensitivity observed in GSK-3β^{-/-} cells may reflect an inability to activate NF-κB. Hence, to determine the degree of TNF-induced NF-κB activation occurring in the absence of GSK-3β, nuclear extracts obtained from EF cell lines treated with TNF-α were analyzed by electrophoretic mobility shift assay (EMSA) using an NF-κB-specific probe. The specificity of the shifted band was tested using a 200-fold excess of non-labelled wild-type or mutant κB DNA (FIG. 4A). Quantitation by phosphorimaging revealed that the NF-κB activation in response to TNF-A treatment was reduced by greater than 50% in GSK-3β^{-/-} cells, as compared to wild-type cells (FIG. 4B). As expected, the NF-κB activity observed in wild-type cells was also sensitive to preincubation with lithium.

Activation of NF-κB was determined by electrophoretic mobility shift assay (EMSA). For oligonucleotide competition assays, wild-type EF cells were incubated with 100 ng/ml TNF-α for 30 min. Equivalent amounts of nuclear extract protein (3 μg) were preincubated for 5 min with a 200-fold excess of either NF-κB-specific oligonucleotide probe containing two tandem NF-κB-binding sites (SEQ ID NO:1 5′-ATC AGG GAC TTT CCG CTG GGG ACT TTC CG-3′ and SEQ ID NO:2 5′-CGG AM GTC CCC AGC GGA MG TCC CTG AT-3′) or mutant NF-κB oligonucleotides (SEQ ID NO:3 5′-GAT CAC TCA CTT TCC GCT TGC TCA CTT TCC AG-3′ and SEQ ID NO:4 5′-CTG GAA AGT GAG CAA GCG CM AGT GAG TGA TC-3′) prior to the addition of radio-labelled NF-κB-specific probe. Reaction mixtures were electrophoresed on 5% nondenaturing polyacrylamide gels containing 6.7 mM Tris (pH 7.5), 1 mM EDTA and 3.3 mM sodium acetate. The position of the activated NF-κB band is indicated. (B) Gel shift analysis of GSK-3β^{+/+} and GSK-3β^{-/-} cells treated with TNF-α. κB-binding activities of cells incubated with lithium or potassium (30 mM, 4 hr) and TNF-α (100 ng/ml, 30 min), as indicated, were compared by EMSA. (C) Immunoblot analysis of cytoplasmic IκB-α and nuclear p65 NF-κB. Cells were stimulated with 10 ng/mL TNF-α for 0, 0.25, 0.5, 1, 2, 4 or 8 hr (lanes 1-7). Nuclear lysates were prepared as follows: cells were disrupted by incubating the monolayer in ice-cold buffer consisting of 10 mM HEPES, pH 7.9, 10 mM KCl, 0.1 mM EDTA, 0.1 mM EGTA and 1 mM DTT, supplemented immediately before use with 50 μg/ml leupeptin, 10 μg/ml aprotinin and 0.5% Nonidet P-40. The disrupted monolayer was mixed in a pipette, transferred to microtubes, and centrifuged at 13,000 rpm for 5 min at 4° C., and the cell pellet was washed with buffer lacking Nonidet P-40 to remove contaminating cytosolic proteins. Nuclear extracts were prepared by vortexing the cell pellets for 30 min at 4° C. in 50 μl of ice-cold buffer consisting of 20 mM HEPES, pH 7.9, 0.4 M NaCl, 1 mM EDTA, 1 mM EGTA, 1 mM DTT, 50 μg/mi leupeptin and 10 μg/ml aprotinin. After centrifugation at 10,000 rpm for 10 min at 4° C., SDS-PAGE and Western blotting were carried out with IκB-A (New England Biolabs) and p65 NF-κB (Santa Cruz Biotechnology) polyclonal antibodies. Non-specific (n.s.) bands are indicated.

### EXAMPLE 5

Kinetics of IκB Proteolysis and NF-κB

In resting cells, NF-κB complexes are retained in the cytoplasm by association with a family of inhibitory proteins termed IκBS. Activation of NF-κB requires the phosphorylation of IκB which triggers its polyubiquitination and subsequent degradation by the 26S proteosome. The liberated NF-κB rapidly translocates to the nucleus where it binds KB sites and regulates gene expression. To assess the mechanism by which NF-κB activity was inhibited in GSK-3β null cells, we studied the kinetics of IκB proteolysis and NF-κB translocation following treatment of cells with TNF-α. Western blotting of cytoplasmic and nuclear extracts indicated no observable differences between wild-type and GSK-3β-deficient cells (FIG. 4C). In all cell lines tested, IκB-α was rapidly degraded following exposure to TNF-α (followed by a lag phase and reaccumulation of the protein by 2 hr). p65 NF-κB translocation to the nucleus was also independent of GSK-3β status. These findings place the defect in NF-κB activation in GSK-3β^{-/-} cells downstream of IκB phosphorylation and nuclear translocation of NF-κB and imply that GSK-3β activity is required for DNA binding of the complex. As has been previously described, partial inhibition of NF-κB DNA binding alone does not seem to be sufficient for down-regulation of the IκB-A gene. Of note, activation of phosphatidylinositol-3-OH kinase (P13K) and PKB/Akt can result in activation of NF-κB as well as partial inactivation of GSK-3.

What is claimed is:

1. A method of inhibiting activity of (nuclear factor-kappa-B) NF-κB in a cell, the method comprising:
contacting said cell with an inhibitor of glycogen synthase kinase-3 (GSK-3) in a dose effective to inhibit said NF-κB activity.

2. The method of claim 1, wherein said GSK-3 is GSK-3β.

3. The method of claim 2, wherein said GSK-3β inhibitor is a direct inhibitor of GSK-3β kinase activity.

4. The method of claim 2, wherein said GSK-3β inhibitor is an indirect inhibitor of GSK-3β kinase activity.

5. The method of claim 2, wherein said GSK-3β inhibitor is an inhibitor of GSK-3β gene expression.

6. The method of claim 2, wherein said GSK-3β inhibitor reduces the level of GSK-3β protein in said cell.

7. The method of claim 2, wherein said NF-κB activity is associated with an inflammatory disease.

8. The method of claim 2, wherein said NF-κB activity is associated with a hyperproliferative disease.

9. The method of claim 8, further comprising:
contacting said cell with a tumor necrosis factor receptor 1 (TNFR1) ligand.

10. The method of claim 9, wherein said TNFR1 ligand is TNF-α.

11. A non-human transgenic animal model for glycogen synthase kinase-3 (GSK-3) gene function wherein the transgenic animal is characterized by having a defect in GSK-3 function.

12. The animal model of claim 11, wherein said GSK-3 is GSK-3β.

13. The animal model of claim 12, wherein the animal is heterozygous for a defect in GSK-3β function.

14. The animal model of claim 12, wherein the animal is homozygous for a defect in GSK-3β function.

15. The animal model of claim 14, wherein said animal is deficient in NF-κB activity.

16. The animal model of claim 12, wherein the defect in GSK-3β function is due to a knockout of GSK-3β expression.

17. A method of screening for biologically active agents that modulate GSK-3β function, the method comprising:
combining a candidate agent with:
a non-human transgenic animal comprising one of: (a) a knockout of an GSK-3β gene; or (ii) an exogenous and stably transmitted mammalian GSK-3β gene sequence; and
determining the effect of said agent on GSK-3β function.

18. A method of screening biologically active agents for the specificity of action on GSK-3β function, the method comprising:
combining a candidate agent with:
a non-human transgenic animal comprising one of: (a) a knockout of an GSK-3β gene; or (ii) an exogenous and stably transmitted mammalian GSK-3β gene sequence; and
determining the effect of said agent on GSK-3β function as compared to an animal comprising normal GSK-3β function.
